(19) United States
(12) Patent Application Publication
(10) Pub. No.: US 2019/0209341 A1
(43) Pub. Date: Jul. 11, 2019

(54) Femoral Head Protection Devices and Methods

(71) Applicant: Michael Gross, Halifax (CA)

(72) Inventor: Michael Gross, Halifax (CA)

(21) Appl. No.: 16/243,804

(22) Filed: Jan. 9, 2019

Related U.S. Application Data
(60) Provisional application No. 62/615,186, filed on Jan. 9, 2018.

(51) Int. Cl. A61F 2/46; A61B 90/00

(52) U.S. Cl. CPC A61F 2/4607; A61F 2002/3611; A61F 2/4637; A61B 90/03

(57) ABSTRACT

Protection devices are described herein. The protection devices include a body having a first end comprising a curved portion integral with the body for engaging a femoral head, the curved portion having a front face that is concave and terminates at an edge extending between a first side of the body and a second side of the body, and a second end integral with and spaced apart from the first end by a middle portion. The second end and the middle portion are generally planar. The front face of the curved portion has a radius of curvature that corresponds to a radius of curvature of an outer surface of the femoral head to guide the femoral head into an acetabular cup. Methods of protecting a femoral head are also described herein.

This application claims the benefit of United Stated Provisional Application Ser. No. 62/615,186 filed Jan. 9, 2018, which is hereby incorporated herein by reference.

### TECHNICAL FIELD

The embodiments disclosed herein relate to protection devices and methods and, in particular to femoral head protection devices and methods.

### BACKGROUND

Total hip replacement, or hip arthroplasty, is a surgical procedure whereby the hip joint is replaced by an articulating surface, typically a ball set on a metal device (e.g. a stem) that is inserted into the thigh bone and a liner of material that is inserted into a metal socket (e.g. a cup) that is inserted into the natural bone socket of the hip (i.e. the acetabulum).

A general movement to improve results of hip arthroplasty has resulted in surgeons devising operations that reduce exposure (i.e. the size and extent of the incisions) during the procedure. Reducing exposure may provide benefits to patients such as less damage to soft tissues around the incisions, decreased blood loss and reduced post-operative pain.

Reducing incision size can decrease the operative view of the acetabulum and the ability for the hip joint to be separated during the procedure, thereby increasing the difficulty of moving the femoral head in and out of the acetabulum. This may increase the likelihood of the femoral head contacting an edge of the new acetabular cup upon insertion into the new acetabular cup.

The acetabular cup edge is typically metal and scratching of and/or metal transfer to the femoral head may occur if the femoral head contacts the acetabular cup edge during insertion. This can result in damage to the femoral head, which can in turn scratch the lining of the acetabular cup and cause premature wear and failure of the replacement.

New operative approaches utilize image intensifiers or mobile x-ray machines in the operating room to show the surgeon the location and position of instruments and prostheses during hip arthroplasty, but they do not often clearly demonstrate the relationship of the femoral head to the acetabulum in all planes.

Accordingly, there is a need for femoral head protection devices, particularly for use during hip arthroplasty.

### SUMMARY

In some embodiments, the protection devices described herein protect the femoral head during hip arthroplasty and inhibit damage to the femoral head.

In some embodiments, the protection devices described herein may provide improved control of the femoral head during insertion of the femoral head into the acetabular cup during hip arthroplasty.

In some embodiments, the devices described herein may interface between the femoral head and the acetabular cup during relocation (e.g. reinsertion) of the femoral head during hip arthroplasty. In some embodiments, when the femoral head is secure within the acetabular cup, the hip joint can be slightly distracted to provide for removal of the device.

In some embodiments, packaging described herein may provide for improved confirmation that portions of the devices described herein have not become separated through use of a highly conforming packaging material indent into which the devices may be placed after use.

In one aspect, a protection device is provided. The protection device includes a body having a first end comprising a curved portion integral with the body for engaging the femoral head, the curved portion having a front face that is concave and terminates at an edge extending between a first side of the body and a second side of the body; and a second end integral with and spaced apart from the first end by a middle portion, the second end and the middle portion being generally planar; wherein the front face of the curved portion has a radius of curvature that corresponds to a radius of curvature of an outer surface of at least a portion of a femoral head to guide the femoral head into an acetabular cup.

In some embodiments, a length of the curved portion is configured to inhibit the curved portion from extending into a deeper side of the acetabular cup when the protection device is inserted into the acetabular cup.

In some embodiments, the curved portion has a depth relative to the middle portion to provide the front face of the curved portion with a degree of curvature substantially the same as a degree of curvature of the femoral head.

In some embodiments, the edge of the curved portion is perpendicular to a length of the body of the protection device.

In some embodiments, the edge of the curved portion has an arcuate shape.

In some embodiments, an angle between a horizontal axis defined by and extending from the middle portion of the protection device and a vertical axis defined by the first edge of the curved portion is in a range of about 0 degrees to about 90 degrees.

In some embodiments, the front face is treated to facilitate sliding of the femoral head along the body.

In some embodiments, the front face is treated with a silicone-based coating.

In some embodiments, the body is made of a resilient material.

In some embodiments, the curved portion has a diameter in a range of about 20 mm to about 50 mm.

In some embodiments, the curved portion has a diameter of about 28 mm, or about 32 mm, or about 36 mm.

According to another aspect, packaging for a protection device is described, the protection device having a body having a first end comprising a curved portion integral with the body for engaging the femoral head, the curved portion having a front face that is concave and terminates at an edge extending between a first side of the body and a second side of the body; and a second end integral with and spaced apart from the first end by a middle portion, the second end and the middle portion being generally planar; wherein the front face of the curved portion has a radius of curvature that corresponds to a radius of curvature of an outer surface of at least a portion of a femoral head to guide the femoral head into an acetabular cup, the packaging having a liner substantially conforming to the protection device.

In some embodiments, the packaging further includes a missing material quality indicator for indicating if a portion of the protection device is missing from the packaging.

In some embodiments, the missing material quality indicator is a floor of the packaging.

In some embodiments, the floor is a different colour than the protection device.

According to another aspect, a method of protecting a femoral head is provided. The method includes inserting a protection device into an acetabular cup, the protection device having a body, the body having a first end comprising a curved portion integral with the body for engaging the femoral head, the curved portion having a front face that is concave and terminates at an edge extending between a first side of the body and a second side of the body; and a second end integral with and spaced apart from the first end by a middle portion, the second end and the middle portion being generally planar; wherein the front face of the curved portion has a radius of curvature that corresponds to a radius of curvature of an outer surface of at least a portion of a femoral head to guide the femoral head into an acetabular cup; sliding the femoral head along the protection device towards the acetabular cup; placing the femoral head within the acetabular cup; and removing the protection device from the acetabular cup.

In some embodiments, the inserting the protection device into the acetabular cup includes inserting an entirety of the curved portion into the acetabular cup.

In some embodiments, the inserting the protection device into the acetabular cup includes inserting the entirety of the curved portion into a portion of the acetabular cup that excludes a deeper side of the acetabular cup.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is an end view of a femoral head protector, according to one embodiment;

FIG. 2 is a top view of femoral head protector shown in FIG. 1;

FIG. 3 is a cross-sectional view of the femoral head protector shown in FIG. 1;

FIG. 4 is a perspective view of the femoral head protector of FIG. 1 inserted into an acetabular cup for protecting the femoral head during reinsertion of the femoral head in hip replacement surgery;

FIG. 5 is a perspective view of the femoral head protector of FIG. 1 inserted into an acetabular cup for protecting the femoral head during hip arthroplasty;

FIG. 6 is a side view of packaging for the femoral head protector of FIG. 1, according to one embodiment

FIG. 7 is a block diagram showing a method for protecting a femoral head, according to one embodiment; and

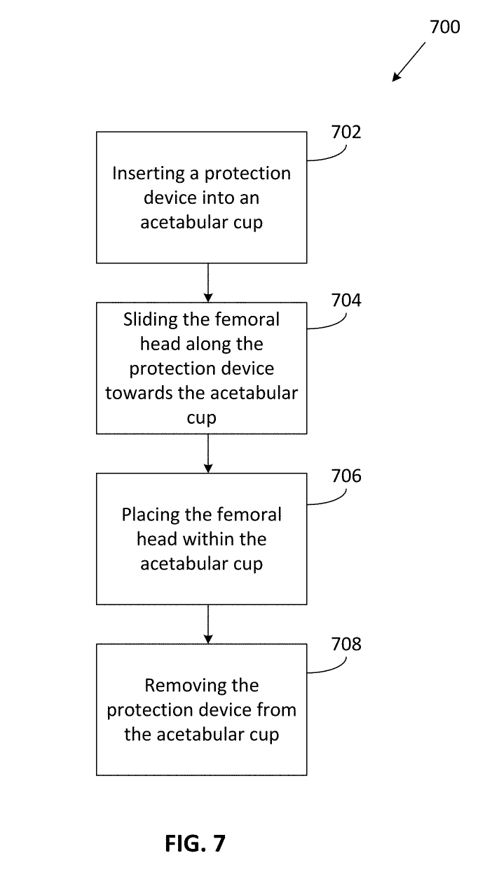

FIG. 8 is a block diagram showing a method for confirming a structural integrity of a protection device, according to one embodiment.

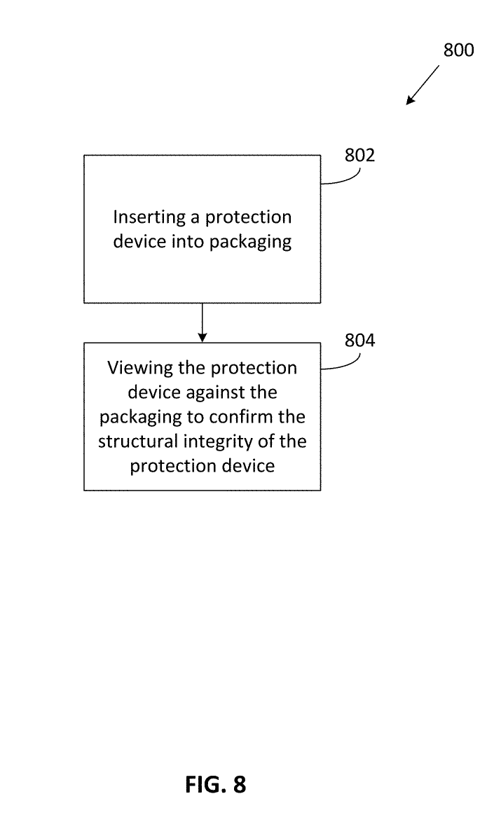

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

Terms of degree such as “about” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

It should be noted that although the embodiments herein refer to use of the protection device 100 during hip arthroplasty specifically, hip arthroplasty is only one example of a surgical procedure where protection device 100 may be used. It is envisioned that protection device 100 may be used in other similar joint replacement procedures and/or other surgical procedures, where similar requirements exist to protect surfaces from edges.

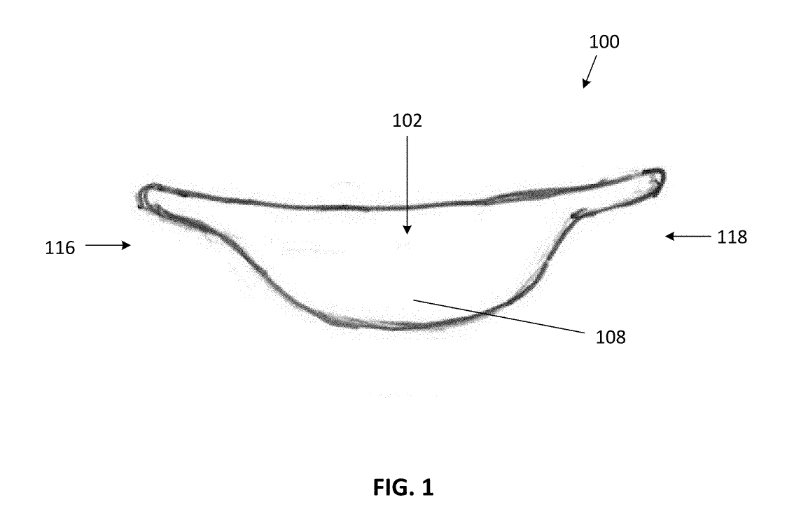

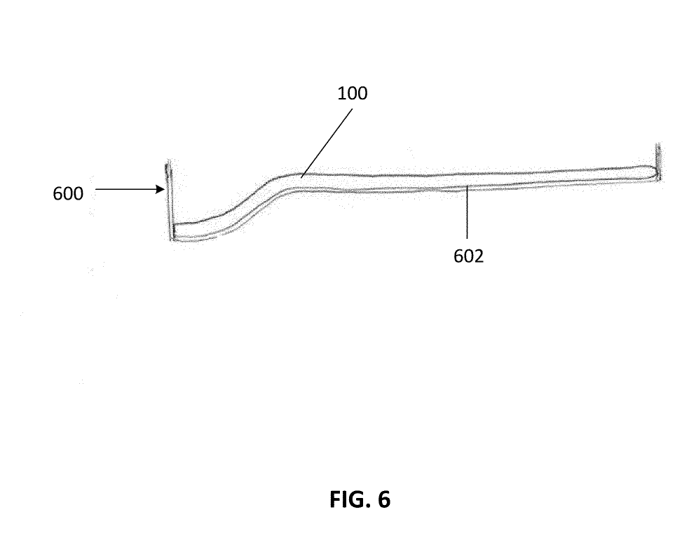

FIGS. 1 to 5 illustrate one embodiment of a protection device 100 for protecting a femoral head (e.g. femoral head 402). FIG. 6 illustrates packaging for the protection device 100.

In one embodiment, protection device 100 comprises an elongate body 102 having opposite first and second ends 104 and 106 spaced apart by a middle portion 105 and an integral curved portion 108 at the first end 104. Curved portion 108 is sized and shaped to engage a head of a femur (i.e. the highest part of the thigh bone; the head is supported by the femoral neck).

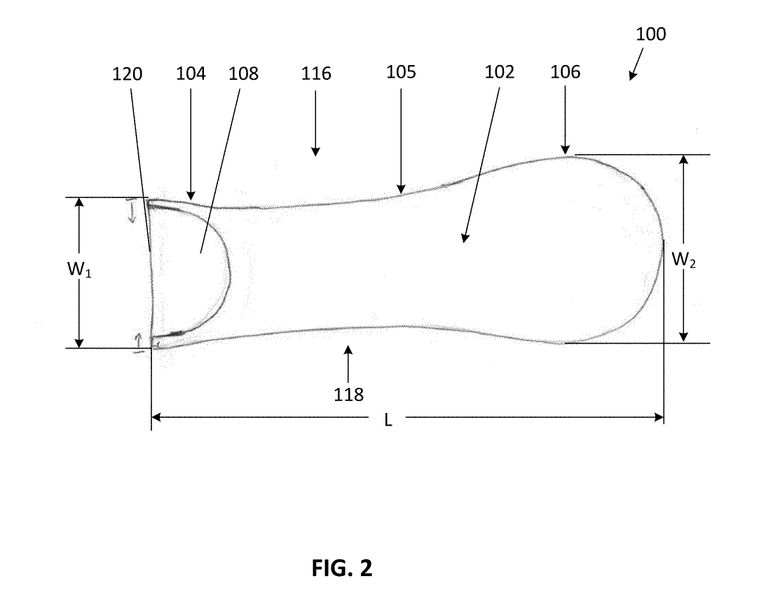

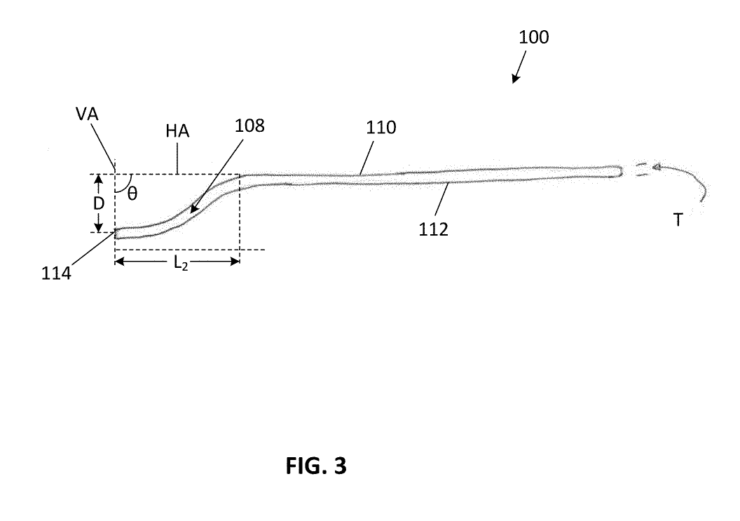

Specifically, FIG. 1 shows an end view of the protection device 100 showing curved portion 108. FIG. 2 shows a top view of protection device 100 illustrating the elongate shape of body 102. Specifically, protection device 100 has a first width W₁ at first end 104, a second width W₂ at second end 106, a length L and a thickness T (see FIG. 3).

The elongate shape of body 102 is generally flat extending from second end 106 through middle portion 105, although the body 102 may have a slight curvature in either a concave or convex fashion. Body 102 generally narrows from second width W₂ as body 102 extends from second end 106 towards middle portion 105 and first end 104, however, second width W₂ and first width W₁ may be the same or second width W₂ may be smaller than first width W₁. Body 102 may also narrow or widen consistently (e.g. linearly) or gradually along length L. In the embodiment shown in the Figures, body 102 narrows from second width W₂ as body 102 extends towards middle portion 105 and first end 104.

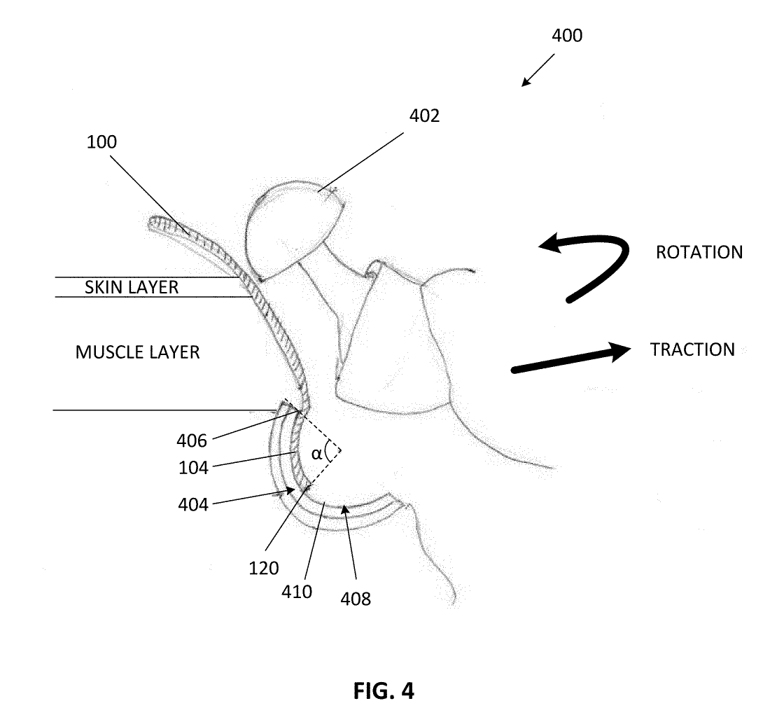

Body 102 is typically uniform in thickness T and has a single body construction (e.g. is made from a single piece of material such as but not limited to a plastic material) that is resilient and may provide for gentle flexibility (e.g. bending) of body 102 generally in middle portion 105. Body 102 is generally made of a material having a rigidity high enough to support the femoral head 402 upon insertion of the femoral head 402 into the acetabular cup (e.g. acetabular cup 404 of FIG. 4), as described below, while protecting the femoral head 402 from damage that may be caused to the femoral head 402 upon contact with the acetabular cup.

As shown in FIG. 3, body 102 has a front face 110 and a rear face 112. At first end 104, specifically at curved portion 108, front face 110 generally has a concave shape. At second end 106 and middle portion 105, front face 110 is generally planar.

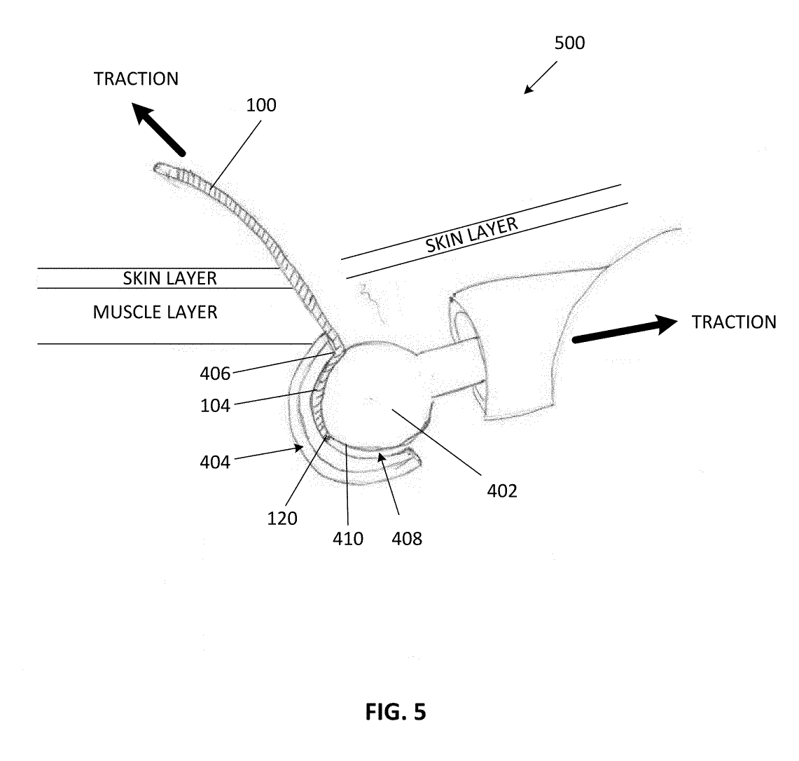

First end 104 terminates at an edge 114 extending between a first side 116 and a second side 118 of protection device 100. Edge 114 can be straight (e.g. substantially perpendicular to elongate body 102), arcuate (e.g. curved), or can be any appropriate shape for providing a terminus (e.g. boundary) of body 102. In one embodiment, edge 114 is sized and shaped to inhibit extension of curved portion 108 into a deeper side 408 of the acetabular cup (see FIGS. 4 and 5) when the protection device 100 is inserted into the acetabular cup during hip arthroplasty. As shown in FIG. 4, the deeper side 408 of the acetabular cup 404 is considered to be the portion of the inner surface 410 of acetabular cup 404 extending beyond an angle α from between an axis extending towards the center 412 of the acetabular cup 404 from edge 408 and an axis extending from the center 412 of the acetabular cup 404 to inner surface 410 of the acetabular cup 404, where the angle α is less than or equal to 90 degrees. In some embodiments, the edge o

Front face 110 extends across body 102 between first end 104 and second end 106 and, in one example embodiment, engages the femoral head 402 of a patient when using protector 100 during hip arthroplasty. At first end 104, front face 110 generally has a concave shape to provide curved portion 108. As shown in FIG. 3, when protection device 100 is in an upright configuration (e.g. where front face 110 is facing upwards), curved portion 108 extends downward from middle portion 105 a distance D to provide curved portion 108 with its concave shape. Curved portion 108 generally has a radius of curvature that is similar to (e.g. mimics) the radius of curvature of at least a portion of an outer surface of a femoral head 402. In some embodiments, the concave shape of curved portion 108 corresponds to at least a portion of a convex shape of an outer surface of a head 402 of a human femur. In some embodiments, a radius of curvature of the curved portion 108 corresponds to a radius of curvature of at least a portion of an outer surface of a head 402 of a human femur to guide the femoral head into an acetabular cup during a hip arthroplasty procedure.

Also shown in FIG. 3, a horizontal axis HA of the protection device 100 is defined by and extends from middle portion 105, a vertical axis VA of the protection device 100 is defined by edge 114 of curved portion 108 and vertical axis VA meets horizontal axis HA at an angle θ. In some embodiments, curved portion 108 extends downward from middle portion 105 a distance D such that angle θ is less than or equal to about 90 degrees. Curved portion 108 has a length L₂ sized to provide for curved portion 108 to extend part way into the acetabular cup when inserted into the acetabular cup. For example, curved portion 108 generally will not extend into deeper side 408 of the acetabular cup (see FIGS. 4 and 5) when the entirety of the curved portion 108 is inserted into the acetabular cup during hip arthroplasty where it may get caught (e.g. upon re-insertion of the femoral head 402 into the acetabular cup during hip arthroplasty) and therefore be difficult to remove.

In some embodiments, front face 110 may be treated (e.g. coated) with a material to facilitate sliding of the femoral head 402 along front face 110 of protection device 100 upon re-insertion of the femoral head 402 into the acetabular cup during hip arthroplasty without damaging (e.g. scratching or scraping) the femoral head 402. In some embodiments front face 110 may be treated with a silicon-based material to provide for the femoral head 402 to slide along front face 110 during re-insertion of the femoral head 402 into the acetabular cup during hip arthroplasty.

Rear face 112 is opposed to front face 110 and generally has a convex shape at curved portion 108. Rear face 112 has a degree of curvature to correspond with the degree of curvature of an inner surface of the acetabular cup.

In some embodiments, curved portion 108 may have a diameter in a range of about 20 mm to about 50 mm, or in a range of about 25 mm to about 40 mm, or be approximately 28 mm, or approximately 32 mm, or approximately 36 mm to provide for placement of curved portion 108 into the acetabular cup.

In some embodiments, curved portion 108 may have a depth D in range of about 10 mm to about 25 mm, or in a range of about 12 mm to about 20 mm, or be approximately 14 mm, or approximately 16 mm, or approximately 18 mm, which correspond to the diameters of approximately 28 mm, approximately 32 mm, or approximately 36 mm above.

In some embodiments, the thickness T of protection device 100 is in a range of about 1 mm to about 10 mm, or in a range of about 1 mm to about 5 mm, or approximately 3 mm.

Referring now to FIGS. 4 and 5, the protection device 100 may be placed into the acetabular cup 404 by inserting concave portion 108 through a wound and laying the curved portion 108 into the acetabular cup 404. It should be noted that herein, the term “traction” is used to refer to the act of pulling something along a surface using motive power, and the term “distraction” is used to refer to the separation of joint surfaces without rupture of their binding ligaments and without displacement.

At 400, the device 100 is shown as being inserted through a wound 402 to provide the first end 104 of the device 100 into the acetabular cup 404.

At 500, traction is applied to the femoral head 402 in direction away from (e.g. as indicated by the arrow in FIG. 5) the protection device 100 to insert the femoral head 402 into the acetabular cup 404.

Curved portion 108 can be led through the wound to a position under the femoral head 402 (as shown in FIG. 4) prior to reduction of the femoral head 402 into the acetabular cup 404. In this manner, the femoral head 402 is encouraged to slide along front face 110 of the protection device 100 during reinsertion. In some embodiments, rear surface 112 of protection device may hold soft tissue (e.g. the skin layer and/or the muscle layer) out of the way, similar to the function of a retractor.

When the femoral head 402 is relocated into the acetabular cup 404, the limb is both distracted to the maximum allowed by the soft tissues and rotated so as to bring the femoral head 402 into the acetabular cup 404. During this step, front face 110 may be treated to slide the femoral head 402 along the front face 410 and aiding the femoral head 402 to follow a path along the protection device and into the indentation.

Upon reinsertion of the femoral head 402 into the acetabular cup 404, the protection device 100 is held in place between the femoral head 402 and acetabular cup 404. At this point, the operator of protection device 100 can pull the femoral head 402 further from the acetabular cup 404 (e.g. in the direction of traction shown in FIG. 5), either by traction on the leg, or by using a bone hook or an equivalent device, to apply traction directly onto the femoral head 402. This maneuver provides for the operator to pull the device out of the joint, so allowing the femoral head 402 to rest back into the acetabular cup 404 once the protection device 100 has been withdrawn and is no longer interposed between the femoral head 402 and the inside of the acetabular cup 404.

Withdrawal of protection device 100 can be aided by the shape of curved portion 108. In some embodiments, curved portion 108 is shaped to cover approximately half of the acetabular cup 404 when the protection device 100 is inserted into the acetabular cup 404. Accordingly, curved portion 108 does not substantially extend to the deeper side 422 of the acetabular cup 404 when inserted into the acetabular cup 404. In this manner, the shape of curved portion 108 of protection device 100 provides for protection device 100 to not become caught in the acetabular cup 404 in a hip replacement where distraction is difficult.

By positioning protection device 100 in the acetabular cup 404, as shown in FIGS. 4 and 5, for example, protection device 100 inhibits contract between the femoral head 402 and an edge 406 of the acetabular cup 404 or other bone fragment of the acetabular cup 404 that could score, scratch and/or otherwise damage the femoral head 402 as the femoral head 402 is relocated into the acetabular cup 404. As noted previously, the shape of curved portion 108 may also facilitate removal of the protection device 100 when the femoral head 402 has been relocated into the acetabular cup 404.

Referring now to FIG. 6, illustrated therein is a side view of packaging 600 for housing protection device 100. In some embodiments, a portion of acetabular cup 404 (e.g. edge 406) may damage (e.g. scratch or scrape) rear face 112 of protection device 100 during use. Accordingly, after use of protection device 100, protection device 100 may be inserted or placed into packaging 600. In some embodiments, packaging 600 comprises a liner (not shown) that substantially conforms to protection device 100, so that if any material (e.g. a portion) of protection device 100 is missing, it is immediately visible as protection device 100 rests in the packaging 600. In other embodiments, a surgeon or other health professional can may also examine the wound to retrieve any material which visible in the wound provided that protection device 100 is generally a bright colour (e.g. blue).

In some embodiments, packaging 600 includes a floor 602 for supporting the protection device 100 when the packaging device is inserted into packaging 600. Floor 602 may be a contrasting colour to the colour of the protection device 100 to provide for visualization of any missing material (i.e. portions) of protection device 100. In this manner, the floor 602 of packaging 600 may be considered as a missing material quality indicator.

In some embodiments, protection device 100 may be used by a surgeon when a soft tissue approach to hip arthroplasty is preferred. In embodiments where the protection device 100 is a bright colour (e.g. blue), protection device 100 may be easily viewable to the surgeon during the procedure.

A hip replacement operation can be accomplished by a variety of surgical approaches through the soft tissues (i.e. muscles, tendons and fat) that surround the hip joint. A surgeon is free to use his/her usual techniques to place the individual components of a hip replacement operation into their desired places in the desired alignments to achieve the desired outcomes. Protection device 100 may be used when the femoral head has been placed onto the femoral neck of either the prosthesis, or the native femoral neck (e.g. when a resurfacing hip arthroplasty has been performed).

In one embodiment of a method of protecting a femoral head 700, at a step 702, before the femoral head 402 is returned into the acetabular cup 404, end 108 of device 100 may be placed into the acetabular cup 404 and the body 102 of the device 100 may be led out of the wound as shown in FIG. 4. At step 704, the surgeon may then initiate a series of manipulations to apply traction to the limb to disengage the femoral head 402 so that the limb can be turned in the direction of the protection device 100 to slide the femoral head 402 down body 102 towards the acetabular cup 404. At step 706, femoral head 402 is then inserted into the acetabular cup 404 where femoral head 402 rests on end 108 of device 100 which is resting within the acetabular cup 404. At step 708, once the surgeon confirms that the femoral head 402 is in place within the acetabular cup 404, either through direct visual inspection or by use of other means such as but not limited to an image intensifier radiological device, the surgeon may then institute a maneuver to distract the femoral head 402 from the device 100 by applying traction to the limb and applying traction to the device 100 to remove it from the acetabular cup 404 (and the wound) (see traction arrows provided in FIG. 5).

In one embodiment of a method of confirming a structural integrity of a protection device disclosed herein 800, following method 700, at step 802, a safety check may be performed whereby the device 100 is inserted (e.g. laid) into the packaging 600 from which it was removed. At step 804, the protection device may be viewed to ascertain that no material (i.e. portion of body 102) is missing or has been removed from the device 100. Once reviewed, a status of completeness may be registered as an event by the circulating nurse in the operative record.

While the above description provides examples of one or more apparatus, methods, or systems, it will be appreciated that other apparatus, methods, or systems may be within the scope of the claims as interpreted by one of skill in the art.

What is claimed is:

1. A protection device comprising:
a body having:
a first end comprising a curved portion integral with the body for engaging the femoral head, the curved portion having a front face that is concave and terminates at an edge extending between a first side of the body and a second side of the body; and
a second end integral with and spaced apart from the first end by a middle portion, the second end and the middle portion being generally planar;
wherein the front face of the curved portion has a radius of curvature that corresponds to a radius of curvature of an outer surface of at least a portion of a femoral head to guide the femoral head into an acetabular cup.

2. The protection device of claim 1, wherein a length of the curved portion is configured to inhibit the curved portion from extending into a deeper side of the acetabular cup when the protection device is inserted into the acetabular cup.

3. The protection device of claim 1, wherein the curved portion has a depth relative to the middle portion to provide the front face of the curved portion with a degree of curvature substantially the same as a degree of curvature of the femoral head.

4. The protection device of claim 1, wherein the edge of the curved portion is perpendicular to a length of the body of the protection device.

5. The protection device of claim 1, wherein the edge of the curved portion has an arcuate shape.

6. The protection device of claim 1, wherein an angle between a horizontal axis defined by and extending from the middle portion of the protection device and a vertical axis defined by the first edge of the curved portion is in a range of about 0 degrees to about 90 degrees.

7. The protection device of claim 1, wherein the front face is treated to facilitate sliding of the femoral head along the body.

8. The protection device of claim 1, wherein the front face is treated with a silicone-based coating.

9. The protection device of claim 1, wherein the body is made of a resilient material.

10. The protection device of claim 1, wherein the curved portion has a diameter in a range of about 20 mm to about 50 mm.

11. The protection device of claim 10, wherein the curved portion has a diameter of about 28 mm, or about 32 mm, or about 36 mm.

12. A packaging for a protection device, the protection device having a body having
a first end comprising a curved portion integral with the body for engaging the femoral head, the curved portion having a front face that is concave and terminates at an edge extending between a first side of the body and a second side of the body; and
a second end integral with and spaced apart from the first end by a middle portion, the second end and the middle portion being generally planar;
wherein the front face of the curved portion has a radius of curvature that corresponds to a radius of curvature of an outer surface of at least a portion of a femoral head to guide the femoral head into an acetabular cup,
the packaging comprising:
a liner substantially conforming to the protection device.

13. The packaging of claim 12, further comprising a missing material quality indicator for indicating if a portion of the protection device is missing from the packaging.

14. The packaging of claim 13, wherein the missing material quality indicator is a floor of the packaging.

15. The packaging of claim 14, wherein the floor is a different colour than the protection device.

16. A method of protecting a femoral head, the method comprising:
inserting a protection device into an acetabular cup, the protection device having a body, the body having
a first end comprising a curved portion integral with the body for engaging the femoral head, the curved portion having a front face that is concave and terminates at an edge extending between a first side of the body and a second side of the body; and
a second end integral with and spaced apart from the first end by a middle portion, the second end and the middle portion being generally planar;
wherein the front face of the curved portion has a radius of curvature that corresponds to a radius of curvature of an outer surface of at least a portion of a femoral head to guide the femoral head into an acetabular cup,
sliding the femoral head along the protection device towards the acetabular cup;
placing the femoral head within the acetabular cup; and
removing the protection device from the acetabular cup.

17. The method of claim 16, wherein the inserting the protection device into the acetabular cup includes inserting an entirety of the curved portion into the acetabular cup.

18. The method of claim 17, wherein the inserting the protection device into the acetabular cup includes inserting the entirety of the curved portion into a portion of the acetabular cup that excludes a deeper side of the acetabular cup.
